(19) United States
(12) Patent Application Publication
(10) Pub. No.: US 2007/0155011 A1
(43) Pub. Date: Jul. 5, 2007

(54) METHOD OF PRODUCING STABLE COLLAGEN PRODUCING MESENCHYMAL CELLS FROM ADULT ADIPOSE STEM CELLS VIA TRANSFECTION

(76) Inventors: Vipul R. Dev, Bakersfield, CA (US); Kevin Slattery, Chandler, AZ (US)

Correspondence Address: James G. O'Neill;Klein, O'Neill & Singh LLP, Suite 204, 43 Corporate Park, Irvine, CA, 92606, US

(21) Appl. No.: 11/619,351

(22) Filed: Jan. 3, 2007

Related U.S. Application Data
(60) Provisional application No. 60/756,016, filed on Jan. 4, 2006.

(30) Foreign Application Priority Data
Dec. 12, 2006 (US) PCT/US06/61938

(51) Int. Cl. C12N 15/87; C12N 5/08

(52) U.S. Cl. 435/440; 435/459; 435/366

(57) ABSTRACT

The present invention provides a method for producing a structurally stable collagen producing mesenchymal pre-adipocyte stem cell and the cells derived therefrom by obtaining fat stem cells from a patient; centrifuging and washing the fat stem cells, culturing the fat stem cells and suspending them in media together with mesenchymal stem cells; and subjecting the fat stem cells to transfection via electroporation using a precisely controlled low voltage, pulsed, electrical field.

### CROSS-REFERENCE TO RELATED APPLICATION

This application claims the priority of co-pending Provisional Application No. 60/756,016, entitled Method of Producing Stable Collagen Producing Mesenchymal Cells from Adult Adipose Stem Cells Via Transfection, filed Jan. 4, 2006, and corresponding PCT Application Number PCT/US2006/061938, filed Dec. 12, 2006.

### BACKGROUND OF THE INVENTION

1. Field of the Invention

This invention relates generally to a method for producing structurally stable collagen producing mesenchymal stem cells and, more particularly, to producing such cells from adult stem cells via transfection facilitated by electroporation.

2. Description of Related Art

A stem cell is an undifferentiated cell found in a differentiated tissue that can renew itself and (with certain limitations) differentiate to yield all the specialized cell types of the tissue from which it originated. Stem cells have two important characteristics that distinguish them from other types of cells. First, they are unspecialized cells that renew themselves for long periods through cell division. The second is that under certain physiologic or experimental conditions, they can be induced to become cells with special functions such as the beating cells of the hear muscle or the insulin-producing cells of the pancreas. In this way, it has been hypothesized by scientists that stem cells may, at some point in the future, become the basis for treating diseases such as Parkinson's disease, diabetes, and heart disease as the healthy cells are transplanted to a diseased patient to replace damaged cells.

Scientists primarily work with two kinds of stem cells in humans: embryonic stem cells and adult stem cells. Human embryonic stem cells are obtained from human embryos, and are thus currently the subject of ethical and political considerations that make them problematic to work with. Adult stem cells, however, avoid such problems and are thus more desirable to work with. Adult stem cells may suffer their own problems, however. Stem cells cultured from one patient may be rejected by another patient, requiring immunosuppressants which ultimately may still be rejected. It would be desirable to transplant autologous cells from a patient, obviating the need for immunosuppression.

Until recently, it had been thought that adult stem cells typically generate the cell types of the tissue in which they reside. A blood-forming adult stem cell in the bone marrow, for example, normally gives rise to the many types of blood cells such as red blood cells, white blood cells and platelets. A blood-forming cell in the bone marrow could not give rise to the cells of a very different tissue, such as the nerve cells in the brain. However, a number of experiments over the last several years have raised the possibility that stem cells from one tissue may be able to give rise to cell types of a completely different tissue. Examples include blood cells becoming neurons, liver cells that can be made to produce insulin, and hematopoietic stem cells that can develop into heart muscle. Therefore, exploring the possibility of using adult stem cells for cell-based therapies has become a very active area of investigation by researchers.

There are several blown methods for transfection to stimulate stem cell differentiation. For example, viral vectors can efficiently transfer genes of interest to a broad range of mammalian cell types. However, despite being derived from non-pathogenic viruses such processes are somewhat controversial. Chemical transfection suffers from detrimental effects of the chemical environment used to insert the gene. Physical transfection can be accomplished with microinjection using a fine microcapillary pipette. Another method involves electroporation which is a well-known technique. The term “electroporation” refers to the use of electric field pulses to induce microscopic pores in the cell membranes called “electropores”. Depending on the parameters of the electric pulse, an electroporated cell can survive the pulse or die. The cause of death of an electroporated cell is believed to be a chemical imbalance, resulting from the fluid communication with the extra cellular environment through the pores. The number and size of electropores created depends on the product of the amplitude E and duration t of the pulse. Below a certain limit, no electropores are induced at all. This limit is different for different cells and depends, principally, on their sizes. The smaller the cell, the higher the product of the amplitude and duration must be to induce pores. Above the lower limit the number of pores and their effective diameter increases with the product Et. Until an upper limit is achieved, cells survive pulsing and restore their viability thereafter. Above the upper limit the pores diameters and number become too large for a cell to survive. It cannot repair itself by any spontaneous or biological process and dies. As noted, a cell's vulnerability to an electric field depends on its size: the larger the cell, the lower the electric field required for killing it.

An article published in the April 2001 issue of Tissue Engineering showed that adipocytes or fat cells taken from liposuction procedures can be utilized as an excellent source of stem cells. Researchers first take the fat and fluid drained from the hips, buttock and stomachs of liposuction patients. The material, referred to as lipoaspirate, is then washed, purified and treated with an enzyme to break down the matrix holding the cells together and compared to stem cells from bone marrow. Scientists found that a half-pound of the fatty substance yielded as many as 50 million to 100 million undifferentiated stem-like cells.

Norbert Wiener in an article in Energy Medicine, entitled The Scientific Basis, coined the term “cybernetics” which is defined as the science of communication and control. The underlying theory advocates that a small energy field applied at the appropriate place and time can shift the course of an organism. This has become a production area of research, for all living processes are ultimately carried out by cells and by the molecules and by the energy fields they produce. Scientists are now learning precisely which steps in the cellular/molecular/electromagnetic cascade are particularly sensitive to exogenous energy fields and which ones are not. They are also discovering how minute signals from the environment are amplified to produce large cellular effects. The significance of cellular amplification was recognized by the 1994 Nobel Price in Physiology of Medicine for the discovery of G-proteins and the role of these proteins in signal transduction in cells.

It is thought that receptors on the cell surface are the primary sites of action of low frequency electromagnetic fields. It is at the receptor site that cellular responses are triggered by hormones, growth factors, nuerotransmitters, light and other electromagnetic signals. Membrane proteins closely associated with receptors, such as adenylate cyclases and G proteins, couple a single molecular event at the cell surface to the influx of a huge number of calcium ions. These calcium ions enter the cell and activate a variety of enzyme molecules. These enzymes in turn act as catalysts and greatly accelerate the biochemical processes. The frequency of the stimulus is crucial. Separate studies of lymphocytes stimulated with a mitogen showed that a weak 3 Hz pulsed magnetic field sharply reduced calcium influx, while a 60 Hz signal, under identical conditions increased calcium influx. New research is revealing how free radicals, including nitric oxide, are involved in the coupling of electromagnetic fields to chemical events in the signal cascade. Again, the medical importance of this research has been recognized by 1998 Nobel Prize in Physiology of Medicine for the discoveries concerning nitric oxide as a signaling molecule in the cardiovascular system.

The human body derives specialized cells from stem cells by specific differentiation signals. For example, biochemical responses are created in cells by applying electromagnetic radiation. A given radiation frequency, or range of frequencies, generates a specific response in each cell. Cell lineages (cartilage, nerve, bone, etc) react to a specific frequency to achieve a specific outcome. That is, cell lineages, are frequency specific for a desired response. Multipotent stem cells can be treated or irradiated with the specific frequency and coaxed or manipulated into differentiating a predetermined cell line. This is accomplished by using pulsed electrical energy to alter the protein (DNA, RNA) sequences which stimulate stem cell differentiation. For example, preadipocytes are the cells that upon receiving specific signals, differentiate into mature adipocytes.

### SUMMARY OF THE INVENTION

It is, therefore, a general object of the present invention to provide a method for producing a structurally stable collagen producing a mesenchymal stem cell and the cells derived therefrom. It is a particular object of the present invention to provide a method for producing a structurally stable collagen producing mesenchymal pre-adipocyte stem cell and the cells derived therefrom comprising the steps of obtaining fat cells from a patient; centrifuging and washing the fat stem cells, culturing the fat stem cells and suspending then in media together with an insulin gene; and subjecting the fat stem cells to transfection via electroporation. And it is a particular object of the present invention to provide a method for producing a structurally stable collagen producing mesenchymal pre-adipocyte stem cell and the cells derived therefrom via transfection that is facilitated by the application of a low voltage, pulsed, electrical field.

### DESCRIPTION OF THE PREFERRED EMBODIMENTS

The following description is provided to enable any person skilled in the art to make and use the invention and sets forth the best modes contemplated by the inventors of carrying out their invention. Various modifications, however, will remains readily apparent to those skilled in the art, since the generic principles of the present invention have been defined herein specifically to provide for an improved method for producing a structurally stable collagen producing mesenchymal pre-adipocyte stem cell and the cells derived therefrom.

More specifically, it is the belief of the inventors that by modifying or controlling a pulsed low voltage electrical field from an electroporation device that is applied to the pre-adipocyte stem cell, we may temporarily create a pore in the stem cell membrane large enough to insert the insulin gene. The low voltage pulse must be strong enough to open the membrane but not strong enough to “kill” the cell. The low voltage pulse must also be weak enough to allow the stem cell membrane to repair itself after transfection of the insulin gene.

In a currently preferred method fat cells to be transfected are obtained from a patient and then centrifuged and washed. The fat stem cells are then cultured and suspended in media together with an insulin gene. The fat stem cells in the media are then subjected to a simple yet versatile electroporator, such as the Gene Pulser® pulse generator from Bio-Rad Laboratories, Inc. of Hercules, Calif. This electroporator or pulse generator having an attached cuvette chamber and simple one-button pulse delivery allows precise control of a wide range of voltage from about 200 to 3,000 volts in 10 volt increments and precise pulse width selection in a 1.0 to 4.0 ms range in 0.1 ms increments.

By precisely controlling the electroporation using the pulse generator, safe and reproducible transformation of adult fat cells is achieved in the attached cuvette.

In the currently preferred embodiment the fat stem cells are taken from a patient and then cultured and suspended in media and placed in the cuvette chamber of an electroporator or pulse generator at an electroporation temperature of 25° C. The cell density should be about 1 to 10×10.6 cells/pulse. The volume of cells is about 500 μl, with the gap of the electrodes in the cuvette about 0.4 cm, the voltage about 0.320 kV, the filed strength about 0.8 kV/cm and the capacitor set at about 500 μF.

After the fat cells are transformed in the cuvette in the pulse generator the transformed cells are recultured in media in a known manner for further use in the patient.

Those skilled in the art will appreciate that various adaptations and modifications of the just-described preferred embodiments can be configured without departing from the scope and spirit of the invention. Therefore, it is to be understood that, within the scope of the appended claims, the invention may be practiced other than as specifically described herein.

What we claim is:

1. A method for producing structurally stable collagen producing mesenchymal pre-adipocyte stem cells comprising:
obtaining fat stem cells from a patient;
centrifuging and washing the fat stem cells;
culturing the fat stem cells and suspending them in media together with an insulin gene; and
subjecting the fat stem cells to transfection via electroporation.

2. The method of claim 1 wherein the transfection is facilitated by the application of a low voltage, pulsed, electrical field.

3. The method of claim 2 wherein the voltage applied ranges from about 200 to 3,000 volts and is applied in pulse widths in a range from about 1.0 to 4.0 ms.

4. The method of claim 3 wherein the cultured fat stem cells in media together with the insulin gene are placed in a cuvette chamber of an a electroporation device at a temperature of about 25° C.

5. The method of claim 4 wherein the fat stem cells density is about 1 to 10×10.6 cells/pulse.

6. The method of claim 5 wherein the fat stem cells volume is about 500 μl.

7. The method of claim 1 wherein the transfection is accomplished by applying a pulsed low voltage electrical field to the fat stem cells that is controlled so as to temporarily create pores in membranes of the fat stem cells large enough to insert an insulin gene.

8. The method of claim 7 wherein the pulsed low voltage electrical field applied is strong enough to open the membranes but not strong enough to kill the fat stem cells and allows the membranes to repair themselves after transfection of the insulin gene.

9. The method of claim 8 wherein the voltage applied ranges from about 200 to 3,000 volts and is applied in pulse widths in a range from about 1.0 to 4.0 ms.

10. The method of claim 9 wherein the fat stem cells are cultured and suspended in media placed in a cuvette chamber of an electroporation device at an temperature of about 25° C.

11. The method of claim 10 wherein the fat stem cells density is about 1 to 10×10.6 cells/pulse.

12. The method of claim 11 wherein the fat stem cells volume is about 500 μl.

13. A method for producing structurally stable collagen producing mesenchymal pre-adipocyte stem cells comprising:
obtaining fat stem cells from a patient;
centrifuging and washing the fat stem cells;
culturing the fat stem cells and suspending them in media together with an insulin gene; and
subjecting the fat stem cells to transfection via electroporation by means of placing the cultured fat stem cells in media together with the insulin gene in a cuvette chamber of an a electroporation device at a temperature of about 25° C.

14. The method of claim 13 wherein the transfection is facilitated by the application of a low voltage, pulsed, electrical field.

15. The method of claim 14 wherein the voltage applied ranges from about 200 to 3,000 volts and is applied in pulse widths in a range from about 1.0 to 4.0 ms; the fat stem cells density is about 1 to 10×10.6 cells/pulse; and the fat stern cells volume is about 500 μl.

16. A method for producing structurally stable collagen producing mesenchymal pre-adipocyte stem cells comprising:
obtaining fat stem cells from a patient;
centrifuging and washing the fat stem cells;
culturing the fat stem cells and suspending them in media together with an insulin gene;
subjecting the fat stem cells to transfection via electroporation; wherein the transfection is facilitated by the application of a low voltage, pulsed, electrical field to the cultured fat stem cells in media together with the insulin gene held in a cuvette chamber of an a electroporation device at a temperature of about 25° C.; and
wherein the voltage applied ranges from about 200 to 3,000 volts and is applied in pulse widths in a range from about 1.0 to 4.0 ms; the fat stem cells density is about 1 to 10×10.6 cells/pulse; and the fat stem cell volume is about 500 μl.
